(19) United States
(12) Patent Application Publication
(10) Pub. No.: US 2020/0391052 A1
(43) Pub. Date: Dec. 17, 2020

(54) BALLOON APPLICATOR FOR DIRECTIONAL INTRAOPERATIVE AND BRACHY RADIATION THERAPY WITH CONFORMAL PHANTOM FOR 3D ANATOMICAL IMAGE REGISTRATION

(71) Applicant: Sensus Healthcare, Inc., Boca Raton, FL (US)

(72) Inventors: Kalman Fishman, Boca Raton, FL (US); James Russell Price, Delray Beach, FL (US); Nicolas Soro, Fort Lauderdale, FL (US)

(21) Appl. No.: 16/901,701

(22) Filed: Jun. 15, 2020

Related U.S. Application Data
(60) Provisional application No. 62/861,782, filed on Jun. 14, 2019.

(51) Int. Cl. A61N 5/10; A61B 34/30

(52) U.S. Cl. CPC A61N 5/1002; A61B 34/30; A61M 25/1018; A61N 2005/1003; A61N 5/1071

(57) ABSTRACT

A balloon applicator for intraoperative radiation therapy including a treatment head includes a connecting sleeve for attachment to the treatment head. The connecting sleeve has proximal and distal ends, the proximal end having an open end for receiving the treatment head. The distal end includes an inflatable balloon contactor for engaging patient tissue during intraoperative radiation therapy. At least one fluid port is provided for supplying and removing inflating fluid to the inflatable balloon contactor. A system and a method for conducting intraoperative radiation therapy are also provided.

### CROSS-REFERENCE TO RELATED APPLICATIONS

This application claims priority to and the benefit of U.S. Provisional Patent Application No. 62/861,782 filed on Jun. 14, 2020, entitled “BALLOON APPLICATOR FOR DIRECTIONAL INTRAOPERATIVE AND BRACHY RADIATION THERAPY WITH CONFORMAL PHANTOM FOR 3D ANATOMICAL IMAGE REGISTRATION”, the entire disclosure of which incorporated herein by reference.

### FIELD OF THE INVENTION

This invention relates to radiation therapy, and more particularly to intraoperative radiation therapy.

### BACKGROUND OF THE INVENTION

X-rays are widely used in the medical field for various purposes, such as radiotherapy. Radiotherapy techniques can involve an externally delivered radiation dose using a technique known as external beam radiotherapy (EBRT). Intraoperative radiotherapy (IORT) is also sometimes used. IORT involves the application of therapeutic levels of radiation to a tumor bed or other target while the area is exposed and accessible during excision surgery. The benefit of IORT is that it allows a high dose of radiation to be delivered precisely to the targeted area, at a desired tissue depth, with minimal exposure to surrounding healthy tissue. The wavelengths of X-ray radiation most commonly used for IORT purposes correspond to a type of X-ray radiation that is sometimes referred to as fluorescent X-rays, characteristic X-rays, or Bremsstrahlung X-rays. Miniature X-ray sources have the potential to be effective for IORT. A challenge with miniature X-ray sources for IORT is that the source is most desirably at least partially positioned within the body of the patient during the IORT procedure, and accordingly portions of the X-ray source assembly come in contact with the patient and must be consumable or capable of resterilization. Proper positioning of the X-ray source relative to the patient tissue and accurate alignment of the X-ray source are required to deliver the X-ray therapy to the patient according to the treatment plan.

### SUMMARY OF THE INVENTION

A balloon applicator for directional intraoperative radiation therapy including a treatment head includes a connecting sleeve for attachment to the treatment head. The connecting sleeve has proximal and distal ends. The proximal end has an open end for receiving the treatment head. The distal end includes an inflatable balloon contactor for engaging patient tissue during intraoperative radiation therapy. At least one fluid port is provided for supplying and removing inflating fluid to the inflatable balloon contactor. The balloon applicator can have at least one inflating fluid supply port and at least one inflating fluid removal port.

The balloon applicator can further include a safety interlock. The safety interlock can be sensible by cooperating structure on the treatment head to signal the presence of the balloon applicator. The safety interlock can be a protrusion for activating a mechanical switch on the treatment head. The safety interlock can be a radio frequency signaling device.

The balloon applicator can include at least one fiducial marker or a conformal fiducial marker phantom for 3D anatomical registration. The balloon applicator can further include an alignment tab for aligning the balloon applicator to the treatment head.

The balloon applicator can have a flange at the proximal end of the connecting sleeve. The flange can include an adhesive for securing the flange to the patient.

A system for conducting intraoperative radiation therapy can include a robotic system for intraoperative radiation therapy comprising a robotic arm secured at a first end to a base, and a treatment head disposed on a second end of the robotic arm distal to the base. The treatment head can include at least one X-ray generation component configured to facilitate generation of therapeutic radiation in the X-ray wavelength range and at lease one X-ray beam forming component for emitting X-rays in a direction selected from a plurality of possible directions in three dimensions.

A balloon applicator for the intraoperative radiation therapy system includes a connecting sleeve for attachment to the treatment head. The connecting sleeve has proximal and distal ends. The proximal end can have an open end for receiving the treatment head. The distal end can have an inflatable balloon contactor for engaging patient tissue during intraoperative radiation therapy. At least one fluid port is provided for supplying and removing inflating fluid to the inflatable balloon contactor. The sleeve of the balloon applicator can be dimensioned to receive the treatment head such that the X-ray beam forming component is positioned in the inflatable balloon contactor.

A method for conducting intraoperative radiation therapy can include the step of providing a robotic system for intraoperative radiation therapy comprising a robotic arm secured at a first end to a base, and a treatment head disposed on a second end of the robotic arm distal to the base. The treatment head comprises at least one X-ray generation component configured to facilitate generation of therapeutic radiation in the X-ray wavelength range and at lease one X-ray beam forming component for emitting X-rays in a direction selected from a plurality of possible directions in three dimensions. A balloon applicator is placed into a target location in a patient's body for the intraoperative radiation therapy. The balloon applicator comprises a connecting sleeve for attachment to the treatment head. The connecting sleeve has proximal and distal ends. The proximal end has an open end for receiving the treatment head. The distal end comprises an inflatable balloon contactor for engaging patient tissue during intraoperative radiation therapy. At least one fluid port is provided for supplying and removing inflating fluid to the inflatable balloon contactor.

The treatment head is placed into the connecting sleeve such that the X-ray beam forming component is positioned within the balloon contactor. An inflating fluid is supplied to the balloon contactor to inflate the balloon contactor and contact patient tissue including target tissue. An X-ray beam is formed with the X-ray generation component and the X-ray beam forming component. The X-ray beam forming component directs an X-ray beam through the balloon contactor to the target tissue. Real-time dose deposition sensors can be embedded within the balloon.

### BRIEF DESCRIPTION OF THE DRAWINGS

There are shown in the drawings embodiments that are presently preferred it being understood that the invention is not limited to the arrangements and instrumentalities shown, wherein:

FIG. 1 is a schematic cross-section of a balloon applicator according to the invention.

FIG. 2A is a plan view. FIG. 2B is a plan view illustrating an alternate flange design.

FIG. 3 is a schematic diagram of a balloon applicator with a balloon contactor in an unexpanded condition.

FIG. 4 is a schematic diagram of a balloon applicator with a balloon contactor in an expanded condition.

FIG. 5 is a schematic diagram of a treatment head with a safety interlock control system.

FIG. 6 is a schematic cross-section of a balloon applicator according to the invention with a treatment head for intraoperative radiation therapy.

FIG. 7 is a block diagram illustrating the operation of a robotic IORT system.

FIG. 8 is a schematic illustration of an implementation of a robotic IORT using a robotic arm to maneuver the treatment head.

FIG. 9 is a schematic diagram illustrating an X-ray beam forming operation in an IORT X-ray source.

FIG. 10 is a schematic perspective view, partially in phantom, of a beam forming component of an IORT.

FIG. 11 is a schematic cross-section of an alternative design of a balloon applicator according to the invention with a treatment head for intraoperative radiation therapy.

### DETAILED DESCRIPTION OF THE INVENTION

A balloon applicator is provided for an intraoperative radiation therapy system which includes a treatment head. The balloon applicator includes a connecting sleeve for receiving and attachment to the treatment head. The connecting sleeve has proximal and distal ends. At the distal end there is an inflatable balloon contactor for engaging patient tissue during intraoperative radiation therapy. At least one fluid port can be provided for supplying and removing inflating fluid to the inflatable balloon contactor. The proximal end of the connecting sleeve can have a locking portion for locking to the treatment head,

The balloon applicator can include a safety interlock. The safety interlock feature is sensible by cooperating structure on the treatment head to signal the presence of the balloon applicator. The safety interlock system includes a control which prevents operation of the treatment head unless properly engaged to the balloon applicator. The safety interlock system can include a protrusion for activating a mechanical switch on the treatment head. Other safety interlock structure as possible, for example, the safety interlock can be a radio frequency signaling device.

Proper positioning of the treatment head relative to the patient tissue that is being treated is vital. The balloon applicator and treatment head are preferably kept to a minimum size. This allows for insertion of the balloon applicator and the treatment head into the surgical opening while keeping the surgical opening as small as possible. Tissue in the body opening will be irregular in shape and consistency. It is desirable to apply a retracting force on the tissue to space the tissue from the treatment head in a controlled manner. This is accomplished by the inflatable balloon contactor.

The balloon contactor is flexible and can be changed from a deflated condition to an inflated condition by the addition of an inflating fluid. The inflating fluid can be a gas or a liquid. Any suitable inflating fluid can be used. A suitable gas is air, and a suitable fluid is water or saline solution. The balloon applicator can have at least one inflating fluid port for the supply and removal of inflating fluid. The balloon applicator can have a separate inflating fluid supply port and an inflating fluid removal port. The inflating supply port communicates with a source of inflating fluid through a inflating fluid supply conduit. The inflating fluid removal conduit can communicate with an inflating fluid removal conduit.

Precise data of the position of the treatment head relative to the balloon contactor and the patient is important for accurate delivery will of radiation therapy. The balloon applicator can comprise at least one fiducial marker to assist in determining the relative position of the treatment head and the balloon contactor. Fiducial marker(s) may be embedded or removable.

The position of the balloon applicator relative to the treatment head can also be monitored by the provision of an alignment indicator to assure proper alignment of the treatment head relative to the balloon applicator. The alignment indicator can be mechanical or a sensor. A suitable mechanical alignment indicator is a protrusion on one of treatment head and the balloon applicator for alignment with a corresponding depression to indicate proper alignment of the treatment head to the balloon applicator.

The balloon applicator can include attachments for maintaining the position of the balloon applicator in the body of the patient during the radiation therapy procedure. In one aspect, the balloon applicator can include a flange at the proximal end of the connecting sleeve. The flange can include an adhesive for securing the flange and thereby the balloon applicator to the patient.

A system for conducting intraoperative radiation therapy includes the balloon applicator and a treatment head for delivering intraoperative radiation therapy. The balloon applicator includes a connecting sleeve for receiving the treatment head. The connecting sleeve has proximal and distal ends. The distal end includes an inflatable balloon contactor for engaging patient tissue during intraoperative radiation therapy. The balloon applicator can have at least one fluid port for supplying and removing inflating fluid to the inflatable balloon contactor. A robotic system for intraoperative radiation therapy can include a robotic arm secured at a first end to a base, and a treatment head disposed on a second end of the robotic arm distal to the base. The treatment head can have at least one X-ray generation component configured to facilitate generation of therapeutic radiation in the X-ray wavelength range and at least one X-ray beam forming component for emitting X-rays in a direction selected from a plurality of possible directions in three dimensions. The sleeve of the balloon applicator can be dimensioned to receive the treatment head such that the X-ray beam forming component is positioned in the inflatable balloon contactor.

A method for conducting directional intraoperative radiation therapy can include the steps of providing a robotic system for intraoperative radiation therapy comprising a robotic arm secured at a first end to a base, a treatment head disposed on a second end of the robotic arm distal to the base, the treatment head comprising at least one X-ray generation component configured to facilitate generation of therapeutic radiation in the X-ray wavelength range and at lease one X-ray beam forming component for emitting X-rays in a direction selected from a plurality of possible directions in three dimensions. A balloon applicator for an intraoperative radiation therapy is positioned in the patient by the target location. The balloon applicator can include a connecting sleeve for receiving the treatment head. The connecting sleeve has proximal and distal ends. The distal end includes an inflatable balloon contactor for engaging patient tissue during intraoperative radiation therapy. The balloon applicator further includes at least one fluid port for supplying and removing inflating fluid to the inflatable balloon contactor. The method includes placing the treatment head into the connecting sleeve of the balloon applicator such that the X-ray beam forming component is positioned within the balloon contactor. Inflating fluid is supplied to the balloon contactor through the inflating fluid port to inflate the balloon contactor and contact patient tissue including target tissue. An X-ray beam is generated with the X-ray generation component and the X-ray beam forming component. The X-ray beam forming component directs an X-ray beam through the balloon contactor to the target tissue. The treatment head can be removed from the balloon applicator. The balloon contactor can be deflated and the balloon applicator removed from the patient's body.

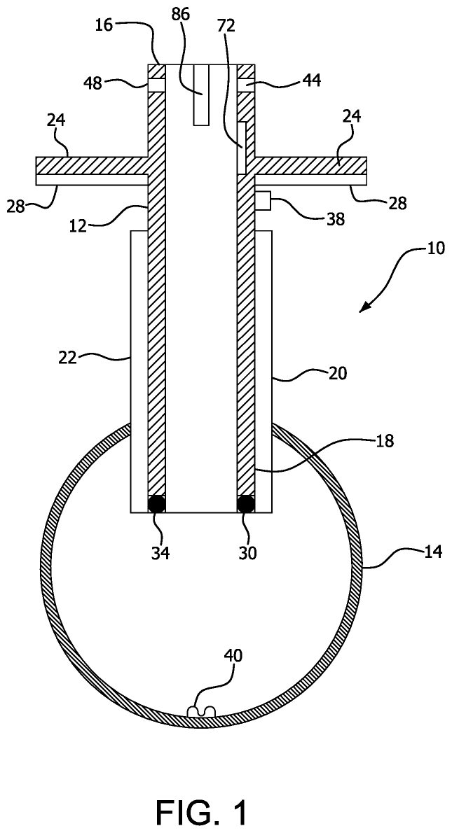

There is shown in FIGS. 1-6 a balloon applicator and system for conducting intraoperative radiation therapy according to the invention. A balloon applicator 10 includes a connecting sleeve 12 and a balloon contactor 14. The sleeve 12 has an open interior and can be generally tubular with a proximal end 16 and a distal end 18. At least the proximal end 16 is open to permit the insertion of a radiation therapy treatment head 54 which is part of radiation therapy treatment system 50. The balloon applicator 10 can also include an inflating fluid supply port 22 and inflating fluid exhaust port 20 for respectively supplying and withdrawing inflating fluid to the balloon contactor 14. The inflating fluid supply port 22 can communicate a inflating fluid supply conduit 23. The inflating fluid exhaust port 20 can communicate with an inflating fluid exhaust conduit 21.

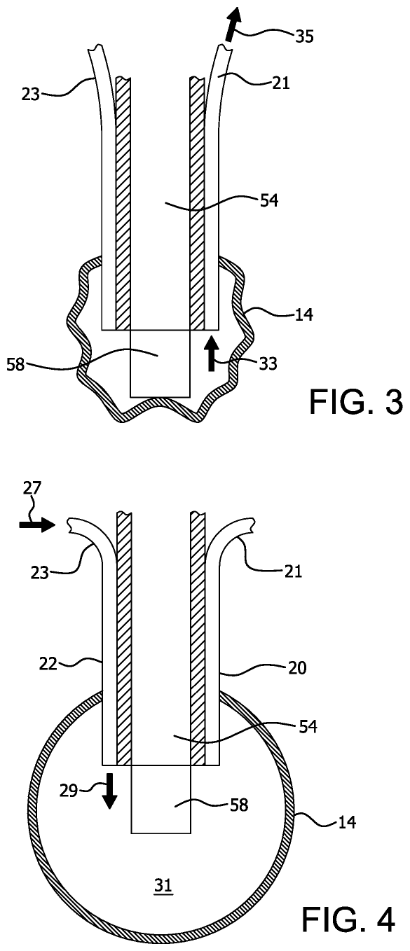

As shown in FIGS. 3-4, the balloon contactor 14 is initially in the deflated condition. Upon receipt of an inflating fluid from inflating fluid supply port 22 and the inflating fluid supply conduit 23 as shown by arrow 27, inflating fluid will enter the interior space 31 of the balloon contactor 14 as shown by arrow 29. The balloon contactor 14 will then expand to the position shown in FIG. 4. Upon completion of the radiation therapy procedure, a suitable pump will be activated to withdraw inflating fluid from the interior space 31 of the balloon contactor as shown by arrow 33. Inflating fluid can be exhausted through the inflating fluid exhaust port 20 and the inflating fluid exhaust conduit 21 as shown by arrow 35 in FIG. 3. The balloon contactor 14 will then return to the deflated condition shown in FIG. 3 such that the balloon applicator 10 can be removed from the patient's body.

Proper positioning of the treatment head 54 and the X-ray beam forming component 58 with end the patient's body is essential for satisfactory radiation therapy. Fiducial markers can be at locations on the balloon applicator 10 to permit sensing by a suitable sensing device such as a radiofrequency sensor such that the position of the balloon applicator 10 and thereby the treatment head 54 and X-ray beam forming component 58 when received in the connecting sleeve 12 of the balloon applicator can be accurately determined. Fiducial markers 30, 34 can be provided at a distal end 18 of the connecting sleeve 12. Fiducial marker 38 can also be provided nearer to or at distal end 16 of the connecting sleeve 12. The fiducial markers can alternatively be located in a fiducial phantom. Fiducial markers can also be provided at one or more locations on the balloon contactor 14, such as fiducial marker 40, to determine the position of the balloon contactor 14, and thus the position of adjacent patient tissue contacting the balloon contactor 14.

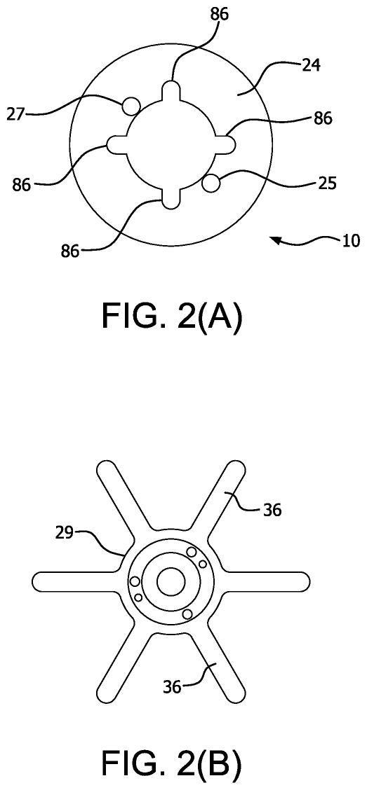

The balloon applicator 10 can also have structure for securing the balloon applicator into proper position relative to the patient's five. A flange 24 can be provided to rest on the patient's body alongside the surgical opening. A mild adhesive 28 can be provided to secure the flange 24 to the patient's body. Apertures 25 and 27 can be provided in the flange 24 to permit passage of the inflating fluid supply conduit 23 and the inflating fluid removal conduit 21 (FIG. 2A). A multiple prong flange design 29 (FIG. 2B) with adhesive prongs 36 can also be utilized.

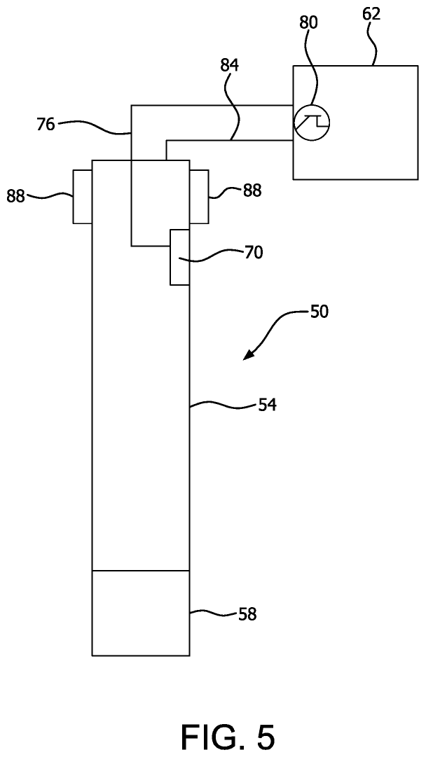

A safety interlock feature can be provided to prevent operation of the treatment head 54 unless it is in proper position within the balloon applicator 10. The treatment head 54 can have a sensor 70 communicating with a processor 62 (FIG. 5). The balloon applicator 10 can have a corresponding sensible device 72 such as metal or RFID to communicate with a sensor 70 when the treatment head 54 is properly within the balloon applicator 10 such that the sensor 70 and sensible device 72 are juxtaposed. A mechanical switch is also possible. A signal line 76 communicates from the sensor 72 to switch 80 which is associated with processor 62. Signal line 76 generates a signal when sensor 70 and sensible device 72 are juxtaposed. Processor 62 responds to the signal through a signal line 84 to permit activation of the treatment head 54 when it is been determined that treatment head 54 is in the proper position relative to the balloon applicator 10.

Alignment of the treatment head 54 with the balloon applicator 10 by the provisions suitable I structure. Different structures are possible. In one aspect, alignment tabs 88 are provided on the treatment head 54. Alignment grooves 86 can be provided on the balloon applicator 10 (FIG. 2). Appropriate alignment of the treatment head 54 with the balloon applicator 10 permits the alignment tabs 88 to move into the alignment grooves 86 to ensure proper positioning of the treatment head 54 relative to the balloon applicator 10. Other alignment structure is possible, such as electronic sensors.

Suitable attachment structure can be provided to secure the treatment head 54 to the balloon applicator 10. Suitable securing structures such as locking screws 45, 47 can be provided in apertures 44, 48 in the connecting sleeve 12 to permit secure attachment of the treatment head 54 and the balloon applicator 10. Other attachment structure is possible.

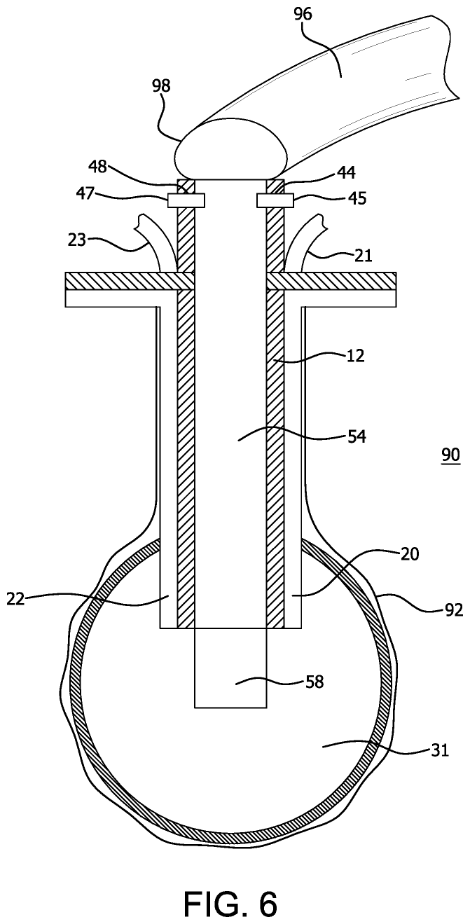

The treatment head 54 is connected to the IORT system through suitable structure such as robotic arm 96 (FIG. 6). A hinge connection 98 can be provided for positioning of the treatment head 54. Movement and operation of the treatment head 54 is controlled by a suitable processor to assure appropriate alignment within the patient's body 90 and the surgical opening 92.

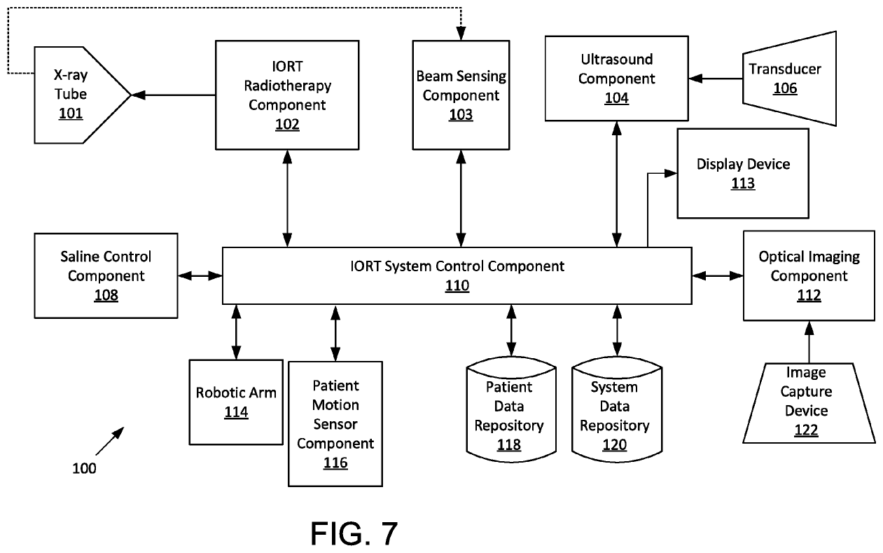

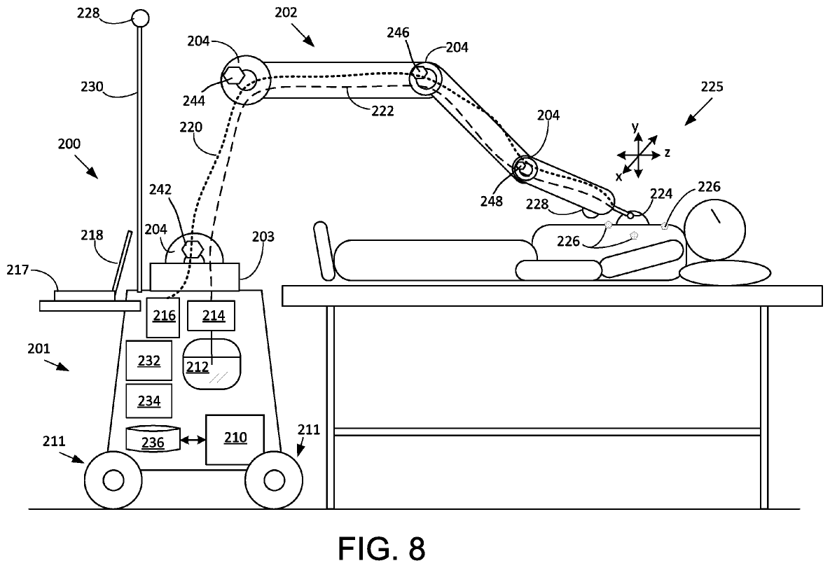

FIG. 7 is a block diagram illustrating an example of the operation of a robotic IORT system. The robotic IORT system 100 can include a radiotherapy component 102 with X-ray tube 101, an optional ultrasound component 104 with a transducer 106, and an optical imaging (01) component 112 with an associated image capture device (ICD) 122. The system can include a robotic arm 114, patient motion sensor 116, and an inflating fluid control component 108 which can be used to control a pump or a manual device such as a syringe. The system control component 110 guides the robotic arm 114 during IORT operations based on images and data obtained from one or more patient motion sensor components 116, the ultrasound component 104, transducer 106, the 01 component 112, and ICD 122. A display device 113, patient data repository 118, and system data repository 120 also can be provided.

An X-ray beam sensing component 103 can monitor beam output from the radiotherapy component and 102 and X-ray tube 101 along with overall system stability and yield. The X-ray beam sensing component 103 can indirectly monitor the performance of the system by determining the characteristics of the X-ray beam that is emanating from the X-ray beam forming component.

When IORT operations are to be performed, the balloon applicator and the treatment head are positioned in the body cavity and the balloon applicator is inflated with fluid. Once inflated, the X-ray tube 101 and radiotherapy component 102 are used to apply radiation to the walls of the cavity formed in the patient. During the application of radiation, the inflating fluid control component can monitor them and maintain fluid circulation and pressure within the balloon. After IORT treatment has been completed, the inflating fluid control component 108 releases the inflating fluid to deflate the balloon and the balloon can be withdrawn from the cavity.

The IORT and x-ray beam forming systems can be any such component for emitting x-rays in a plurality of possible directions in three dimensions. Preferably, the beam forming component can selectively emit x-rays in any of a plurality of possible directions in three dimensions, while selectively excluding emissions in some directions. One such system is shown in US 2018/0286623 dated Oct. 4, 2018 “THREE-DIMENSIONAL BEAM FORMING X-RAY SOURCE”, the disclosure of which is incorporated fully herein by reference. The intraoperative radiation therapy system can be any of several possible designs. One such system is shown in US 2018/0015303 dated Jan. 18, 2018 “ROBOTIC INTRAOPERATIVE RADIATION THERAPY” the disclosure of which is incorporated fully herein by reference.

FIG. 8 is a schematic illustration of an implementation of a robotic IORT using a robotic arm in the treatment head. The robotic IORT system 200 can include a base unit 201 and a robotic arm 202, radiotherapy treatment device 216, inflating fluid reservoir 212, an inflating fluid control element 214, and a system control component 210. The base unit 201 can be mounted on wheels 211 to provide mobility. The base unit can also include an optical imaging component 232, an ultrasound component 234, and a data storage device 236 for storing patient and/or system data. The base unit 201 can include a power lead for optionally providing power to all the components housed in or connected to the base unit 201. The base unit 201 can contain one or more computers 217 for controlling the system 200 and/or analyzing and processing data obtained from the system 200 components. A monitor 218 can also be mounted to the base unit 201 for user interface. A terminal or an input device such as a keyboard or mouse can also be included. Fiducial markers 226 can be provided and monitored by sensors 228 and optionally sensing support structure 230 can be provided.

A mount 203 is provided on the base unit 201 for mounting the robotic arm 202. The robotic arm 202 can include a treatment head 224 which can include removable and replaceable balloon applicators of the invention for beam hardening the applied IORT. The robotic arm 202 is articulated with appropriate robotic joints or articulation members 204 under the control of the system control component 210. Additional articulations can also be provided different points of robotic arm 202 to increase the number of degrees of freedom 225 of placing, orienting and moving treatment head 224. An inflating fluid conduit 222 can facilitate communication of inflating fluid from the reservoir 212 and inflating fluid control component 214 to the treatment head 224. Power and/or control signals can be communicated from the radiotherapy treatment device 216 to the treatment head 224 by control line 220 to control and facilitate operation of the X-ray tube. The force of patient tissue movement exerted on the treatment head can be sensed by physical sensors 242, 244, 246, and 248 located in any of several positioned throughout the robotic arm 202.

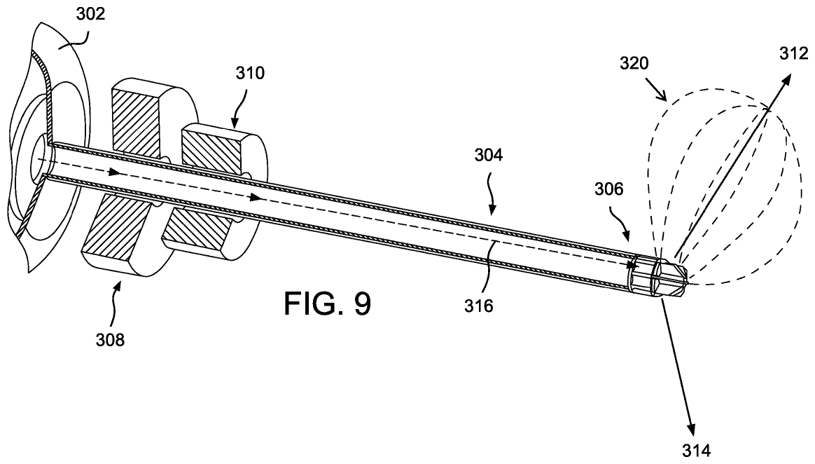

The invention can be utilized with different X-ray beam generating equipment for the treatment head. FIG. 9 is a schematic diagram illustrating an X-ray beam forming operation in an IORT X-ray source that is capable of emitting X-ray beams in three dimensions. The treatment head 304 includes a beam directionally controlled target assembly (DCTA) 306 comprising the X-ray source, beam focusing unit 308 and a beam steering unit 310. An envelope 302 encloses a vacuum chamber. An X-ray beam can be aligned in a plurality of different directions 312, 314 by selectively controlling the electron beam 316. The exact three-dimensional shape or relative intensity pattern of the X-ray beam 320 will vary in accordance with several factors. In some scenarios, the electron beam can be rapidly steered so that different target segments are success of the successively bombarded with electron so that the electron beam intersex different target segments for predetermined dwell times. If more than one target segment is bombarded by the electron beam, then multiple beam segments can be formed in selected directions defined by the associated beam-formers and each can have a different beam shape or pattern.

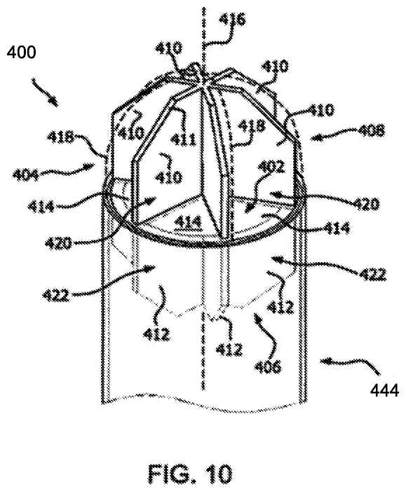

The invention can be utilized with different bean forming designs. FIG. 10 is a schematic perspective view, partially in phantom, of a directionally controlled target assembly (DCTA) or beam forming component for the X-ray source of an IORT 400. Other designs for a DCTA or beam forming component are possible. The beam forming component is comprised of a target 402 and a beam shield 404. The target 402 is comprised of a disk-shaped element, which is disposed transverse to the direction of electron beam travel. The beam shield 404 can include a first portion 406 which is disposed adjacent to one major surface of the target 402, and a second portion 408, which is disposed adjacent to an opposing major surface of the target. In some scenarios, the first portion 406 can be disposed internal of the drift tube 444 within a vacuum environment, and the second portion 408 can be disposed external of the drift tube. If a portion of the beam shield 404 is disposed external of the drift tube then an X-ray transmissive cap member 418 can be disposed over the second portion 408 of the beam shield to enclose and protect the portions of the DCTA external of the drift tube. The cap member is indicated by dotted lines and it should be understood that the cap member 418 would extend from the end of the drift tube 444 so as to enclose the first portion 406 of the DCTA.

The beam shield 404 is comprised of a plurality of wall elements 410, 412. The wall elements 410 associated with the first portion 406 can extend from a first major surface of the disk-shaped target which faces in a direction away from the electron beam generator. The wall shaped elements 412 associated with the second portion 408 can extend from the opposing major surface of the target facing toward the electron beam generator. The wall elements 410, 412 also extend in a radial direction outwardly from the centerline 416 toward a periphery of the disk-shaped target 402. Accordingly the wall elements form a plurality of shielded compartments 420, 422. The wall elements 410, 412 can be advantageously comprised of the material which interacts in a substantial way with X-ray photons. In some scenarios, the material can be one interacts with the X-ray photons in a way which causes the X-ray photons to give up a substantial part of its energy and momentum. Accordingly one type of suitably interactive material for this purpose can comprise material that it attenuates or absorbs X-ray energy. In some scenarios the material chosen for this purpose can be advantageously chosen to be one that is highly absorbent of X-ray energy.

Suitable materials which are highly absorptive of X-ray radiation are well known. For example, these materials can include certain metals such as stainless steel, molybdenum (Mo), tungsten (W), tantalum (Ta), or other high atomic number (high-Z) materials. As used herein the phrase high-Z material will generally include those which have an atomic number of at least 21. There may be some scenarios in which a lesser degree of X-ray absorption is desired. In such scenarios a different material may be suitable. Accordingly, a suitable material for the shield wall is not necessarily limited to high atomic number materials.

The plurality of wall elements extend radially outward from the centerline 416. However the configuration of the beam shield is not limited in this regard and it should be understood that other beam shield configurations are possible. Several of such alternative configurations are described below in further detail. Each of the wall elements can comprise rounded or chamfered corners 411 to facilitate beam formation as described below. The rounded or chamfered corners can be disposed at portions of the wall elements, which are distal from the target 402 and spaced apart from the centerline 416.

The wall elements 410 can be aligned with wall elements 412 to form aligned pairs of shielded compartments 420, 422 on opposing sides of the target 402. Each such shielded compartment will be associated with a corresponding target segment 414 which is bounded by a pair of wall elements 410 on one side of the target 402, and a pair of wall elements 412 on an opposing side of the target.

As is known, X-ray photons are released in directions which are generally transverse to the collision path of the electron-beam with the major surface of the target 402. The target material is comprised of a relatively thin layer of target material such that electrons bombarding the target 402 produce X-rays in directions extending away from both major surfaces of the target. Each aligned pair of shielded compartments 420, 422 (as defined by wall elements 410, 412) and their corresponding target segment 414 comprise a beam-former of the beam forming component. X-rays which are generated in high-energy electrons interact with a particular target segment 414 will be limited in their direction of travel by the wall elements defining the compartments 410, 412.

An electron-beam bombards a segment of target 402 to produce transmitted and reflected X-rays in directions that are generally transverse to the collision path of the electron beam. However, the X-rays will only be transmitted over a limited range of azimuth and elevation angles α, β due to the shielding effect of the beam-former. By selectively controlling which target segment 414 is bombarded with electrons, and where within the target segment 414 that the electron-beam actually strikes the target segment, the X-ray beams in a range of different directions and shapes can be selectively formed and sculpted as needed.

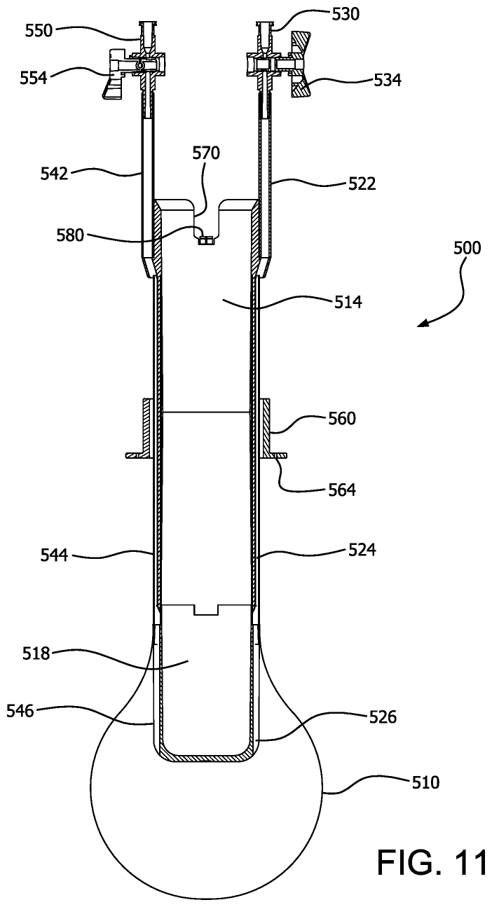

There is shown in FIG. 11 another design 500 of the balloon applicator as positioned over a treatment head 514 having an x-ray beam forming component 518. An inflating fluid supply conduit 522 can communicate with an inflating fluid inlet conduit 524 and inflating fluid inlet opening 526 to deliver inflating fluid to the balloon 510. An inlet port 530 can be controlled by suitable structure such as valve 534 to control the supply of inflating fluid and to retain the inflating fluid within the balloon contactor 510. A syringe or other supply source can be positioned in or connected to the port 530 to deliver inflating fluid to the balloon applicator 500. An inflating fluid exhaust conduit 542 can communicate with an inflating fluid outlet conduit 544 and inflating fluid outlet opening 526 to withdraw inflating fluid from within the balloon contactor 510. An outlet or exhaust port 550 can be controlled by suitable structure such as valve 554 to control the withdrawal of inflating fluid and also to retain the inflating fluid within the balloon contactor 510 during operation of the balloon applicator. A syringe or other device can be positioned in or connected to the port 550 to withdraw inflating fluid from within the balloon contactor 510. A sheath 560 and flange 564 can be provided for positioning the balloon applicator on the patient. An alignment tab 570 and safety switch contact 580 for indicating the presence or absence of the balloon applicator 500 over the treatment head prior to operation can also be provided.

This invention can be embodied in other forms without departing from the spirit or essential attributes thereof. Accordingly, reference should be made to the following claims to determine the scope of the invention.

We claim:

1. A balloon applicator for directional intraoperative radiation therapy including a treatment head, comprising:
a connecting sleeve for attachment to the treatment head, the connecting sleeve having proximal and distal ends, the proximal end having an open end for receiving the treatment head, the distal end comprising an inflatable balloon contactor for engaging patient tissue during intraoperative radiation therapy;
at least one fluid port for supplying and removing inflating fluid to the inflatable balloon contactor.

2. The balloon applicator of claim 1, further comprising a safety interlock, the safety interlock being sensible by cooperating structure on the treatment head to signal the presence of the balloon applicator.

3. The balloon applicator of claim 2, wherein the safety interlock is a protrusion for activating a mechanical switch on the treatment head.

4. The balloon applicator of claim 2, wherein the safety interlock is a radio frequency signaling device.

5. The balloon applicator of claim 1, further comprising at least one fiducial marker or a conformal fiducial marker phantom for 3D anatomical registration.

6. The balloon applicator of claim 1, further comprising an alignment tab for aligning the balloon applicator to the treatment head.

7. The balloon applicator of claim 1, further comprising a flange at the proximal end of the connecting sleeve.

8. The balloon applicator of claim 7, wherein the flange comprises an adhesive for securing the flange to the patient.

9. The balloon applicator of claim 1, comprising at least one inflating fluid supply port and at least one inflating fluid removal port.

10. A system for conducting intraoperative radiation therapy, comprising:
a robotic system for intraoperative radiation therapy comprising a robotic arm secured at a first end to a base, a treatment head disposed on a second end of the robotic arm distal to the base, the treatment head comprising at least one X-ray generation component configured to facilitate generation of therapeutic radiation in the X-ray wavelength range and at lease one X-ray beam forming component for emitting X-rays in a direction selected from a plurality of possible directions in three dimensions;
a balloon applicator for intraoperative radiation therapy including a treatment head, comprising a connecting sleeve for attachment to the treatment head, the connecting sleeve having proximal and distal ends, the proximal end having an open end for receiving the treatment head, the distal end comprising an inflatable balloon contactor for engaging patient tissue during intraoperative radiation therapy, and at least one fluid port for supplying and removing inflating fluid to the inflatable balloon contactor;
the sleeve of the balloon applicator being dimensioned to receive the treatment head such that the X-ray beam forming component is positioned in the inflatable balloon contactor.

11. A method for conducting intraoperative radiation therapy, comprising the steps of:
providing a robotic system for intraoperative radiation therapy comprising a robotic arm secured at a first end to a base, a treatment head disposed on a second end of the robotic arm distal to the base, the treatment head comprising at least one X-ray generation component configured to facilitate generation of therapeutic radiation in the X-ray wavelength range and at lease one X-ray beam forming component for emitting X-rays in a direction selected from a plurality of possible directions in three dimensions;
placing into a target location in a patient's body a balloon applicator for intraoperative radiation therapy, the balloon applicator comprising a connecting sleeve for attachment to the treatment head, the connecting sleeve having proximal and distal ends, the proximal end having an open end for receiving the treatment head, the distal end comprising an inflatable balloon contactor for engaging patient tissue during intraoperative radiation therapy, and at least one fluid port for supplying and removing inflating fluid to the inflatable balloon contactor;
placing the treatment head into the connecting sleeve such that the X-ray beam forming component is positioned within the balloon contactor;
supplying inflating fluid to the balloon contactor to inflate the balloon contactor and contact patient tissue including target tissue;
forming an X-ray beam with the X-ray generation component and the X-ray beam forming component, the X-ray beam forming component directing an X-ray beam through the balloon contactor to the target tissue.

12. The method of claim 11, comprising real-time dose deposition sensing embedded within the balloon.
